(19) United States
(12) Patent Application Publication
(10) Pub. No.: US 2018/0356397 A1
(43) Pub. Date: Dec. 13, 2018

(54) MIMICRY OF NEUROINFLAMMATORY MICROENVIRONMENTS AND METHODS OF USE AND MANUFACTURING THEREOF

(71) Applicant: Hansang Cho, Charlotte, NC (US)

(72) Inventor: Hansang Cho, Charlotte, NC (US)

(21) Appl. No.: 15/670,354

(22) Filed: Aug. 7, 2017

Related U.S. Application Data
(60) Provisional application No. 62/516,736, filed on Jun. 8, 2017.

(51) Int. Cl. G01N 33/50

(52) U.S. Cl. CPC G01N 33/5029; G01N 33/5088

(57) ABSTRACT

A method for simulating the neuroinflammatory response in brains comprising the steps of providing a microfluidic device for regulating microgliosis comprising a base, a central chamber located on the base, a size-exclusive barrier surrounding the central chamber, an annular chamber outside the barrier, a plurality of selective cellular migration channels connecting the annular chamber to the central chamber, a central reservoir in fluid communication with the central chamber and side reservoirs in fluid communication with the annular chamber; providing a camera to record the microfluidic device and cells, culturing a plurality of microglial cells within the central or annular chamber, culturing a plurality of a second cell type within the annular or central chamber, adding a chemoattractant to the central chamber and recording the microfluidic device for a period of time to record the morphogenesis of activated cells in the annular chamber and migration across the barrier.

### RELATED CASES

This application claims the priority of the provisional application Ser. No. 62/516,736 filed Jun. 8, 2017. Applicant hereby incorporates by reference the entire content of provisional application Ser. No. 62/516,736.

### APPENDIX

The present application contains an appendix labeled as “Appendix-A”. The entire contents of which are hereby incorporated by reference.

### FIELD OF THE INVENTION

The present invention relates to the regulated neuroinflammatory microenvironments and more specifically to an in vitro microglial model.

### BACKGROUND OF THE INVENTION

Alzheimer's disease (AD) is the leading cause of age-related neurodegeneration affecting over 5.2 million people in the United States alone. While our knowledge regarding the mechanisms underlying the AD pathogenesis has greatly improved in recent decades, there is no cure. Moreover, many questions remain regarding pathogenic cascades of AD including the mechanisms underlying synaptic loss, axonal damage and neuronal death. This involves complex and multistep processes guided by a wide spectrum of genetic determinants and the only clinical approach currently available is analysis of postmortem brain tissue. Alzheimer's disease is an irreversible neurodegenerative illness with long preclinical, prodromal phases (20 years), and an average clinical duration of 8 to 10 years. The disease has an estimated prevalence of 10-30% in the population >65 years of age with an incidence of 1 to 3 percent. Most AD patients (>95%) have the sporadic form, which is characterized by a late onset (80-90 years of age). The developing AD stage is characterized with an accumulation of amyloid-beta (A-beta) peptides, extracellular amyloid plaques in the brain parenchyma, and the formation of intracellular tangles within neurons as a result of abnormal phosphorylation of the microtubule-associated tau proteins. Consequently, prominent activation of innate immune cells is observed and followed by marked neurotoxic neuroinflammation. While such responses may be generated to clear neurotoxic reagents and protect the host, neuroinflammation may be detrimental to the subject because of the severe disease process and driving pathology. The hallmarks of AD include elevated levels of neurotoxic proinflammatory mediators, decreased levels of neurotransmitters, synaptic and cognitive impairment, neuronal death, reduced memory, and brain malfunctions initiating from the neocortex and hippocampus. Microglia, resident myeloid cells in the CNS, continually survey their microenvironments in normal and diseased brains while providing immune surveillance and activation in response to infection, non-infectious diseases, and injury. Recent findings suggest that binding of A-beta to specific receptors on microglia can trigger neuroinflammation, with chronic deposition of A-beta stimulating microglial-mediated neuroinflammatory responses and augmenting neurodegeneration by impairing synaptic function in AD brains.

Microglia (i.e., resident myeloid cells in the CNS) continually survey their microenvironments in both normal and diseased brains while providing immune surveillance and activating in response to infectious and non-infectious diseases and injury. In addition to A-beta plaque and tau neurofibrillary tangle deposition, neuroinflammation is considered a key feature of AD pathology. Upon activation, microglia rapidly change their morphology and phenotypes, expressing an array of receptors and mediators in response to CNS disturbances. Inflammation in AD is characterized by the presence of reactive astrocytes and activated microglia surrounding amyloid plaques, implicating their role in disease pathogenesis. Hong, et al. recently reported that microglia mediate synaptic loss early in AD mediated with C1q, necessary for the toxic effects of soluble A-beta oligomers on synapses and hippocampal long-term potentiation (LTP). At the late stage of AD, microglia in adult brains engulf synaptic material in a CR3-dependent process when exposed to soluble A-beta oligomers, suggesting that the complement-dependent pathway and microglia that prune excess synapses in development are inappropriately activated and mediate synapse loss in AD. Bialas et al. investigated the brain inflammation that can occur as a feature of an autoimmune disease called lupus. The authors used previously established mouse having a higher level of interferon than do wild-type animals. They observed that the microglial cells in the model mice adopted an activated state that involved activation of immune functions, including the ingestion of cellular material through a process known as engulfment. Using image analysis, the authors observed that the activated microglial cells ingested synaptic material from neurons, resulting in a reduced synaptic density compared with that of wild-type animals. Collectively, these results demonstrate that microglia contribute to neuronal loss and memory impairments in AD mice. Therefore, it is widely accepted that stimulated microglial neuroinflammatory responses may augment neurodegeneration in AD.

Therefore, the next challenge is the generation of a human organotypic neurodegeneration model that includes neuroinflammation, a key component of all neurodegenerative disorders commonly induced through activation of microglia. The sustained activation of microglia results in a chronic neuroinflammatory response and increased production of proinflammatory cytokines, such as TNF-α and IL-1β. Of relevance, recent genome-wide association studies have uncovered several risk-associated genes in the development of sporadic AD. Most of these risk genes are either expressed by microglia or associated with their reactivity, including CD33, BIN1, CR1, TREM2, and CLU. As such, there is a pressing need for the development of organotypic cell culture platforms that include multiple brain cell types to detail cellular pathogenic mechanisms and, possibly, recapitulate the complex cell-cell interactions and microenvironment associated with AD more accurately.

Currently, animal studies are an integral part of drug development and toxicology evaluation. Each year, hundreds of millions of animals are used for animal studies. It is expensive, cumbersome and ethically controversial. Furthermore, there are concerns for extrapolating the data from animal studies to be used in humans. Hence, there is a need in finding alternatives to animal studies that are cheaper, faster, more humane, and capable of achieving more accurate results. There is a crucial need for alternatives to animal studies for understanding and eventually treating neurodegenerative diseases. One approach to replace or reduce reliance upon animal studies is to replicate tissue and organ-level functions in vitro. Living organs are three-dimensional vascularized structures composed of two or more closely apposed tissues that function collectively and transport materials, cells and information across tissue-tissue interfaces in the presence of biological/chemical/mechanical cues. The development of microfluidic devices containing living human cells cultured within that recapitulate the three-dimensional (3D) tissue-tissue interfaces, mechanically active microenvironments, electrical stimulation, chemical conditions and complex organ-level functions is progressing rapidly. The new microfluidic devices can shorten the drug development timeline, save animal lives, reduce failure rates, inform regulatory decision-making, and accelerate development of new therapeutics in neuroscience.

Hence, a need exists to develop a viable and reliable method for simulating a neuroinflammatory response in human brains to permit experimentation and visualization of a variety of multicellular interactions to better understand disease development and progression.

### SUMMARY OF THE INVENTION

Activated microglia initially take on various morphologies: rounded, ramified shapes, rods to amoeboid forms followed by motile activation, which complicates the visual tracking of individual cells. Previous in vitro attempts to study rat microglial migration in the presence of short-lived damaged axons could not establish long-lasting gradients and could not conclusively differentiate slow accumulation of microglia from heterogeneous activation and random navigation. We developed a novel, microfluidic chemotaxis platform to study the motility of those microglia selectively responding to stimuli in a regulated manner. To understand the specific roles of A-beta in microglial accumulation in the context of AD, we generated a week-long lasting soluble A-beta gradient, patterned insoluble surface-bound A-beta to mimic A-beta signature in AD brains, and isolated human primary microglial responding to A-beta in our platforms. We tested 200 independent conditions in a single batch, while using real time imaging of process in an array of twenty-five formatted single well plate. In addition, we could monitor single cellular microglial responses as morphological changes in response to soluble A-beta that resulted in directional migration in real time. We found a broad range of soluble monomeric and oligomeric A-beta from picomolar to nanomolar concentrations, that correspond to levels in normal and AD brains, respectively, acting as a microglial chemoattractant. We also observed discernable co-localization of microglia to the sites of insoluble A-beta, representing A-beta plaques.

In contrast to the field's increasing understanding of the inflammatory role of innate immunity—microglia in AD, comparatively little is known about their interaction with the peripheral immune system. Peripheral immune cells including lymphocytes, T cell, B cell, monocytes and neutrophils, have been identified in the brains of human patients with AD and in corresponding animal models. Neutrophils are known primary effectors of inflammatory response. This function relies on the neutrophil's remarkable ability to migrate within and through blood vessels. Here, we reconstituted an AD microenvironment: induction of neuroinflammation by stimulating human microglia with amyloid-beta (A-beta), a signature molecule in AD, and employed this environment to investigate the recruitment mechanism of human peripheral immune cells—neutrophil—in the context of the innate-peripheral immunity crosstalk between peripheral immune system and microglia in the brain. To accommodate the different cells separately in a single microfluidic platform, we employed mechanically separated two chambers, which are connected through neutrophil specific migration channels 60 (10×5×500 μm in width, height, and length) forming gradients of chemokines and/or cytokines. The long and thin migration channels can block spontaneous entrance of inactivated neutrophil through mechanical constraints. Using this platform, we could culture microglial cells for extended periods to accumulate enough soluble factors and could observe neutrophil signature movement at single cell resolution, in real-time. Red membrane dye stained microglial cells plated first in central chamber and incubated for 3 days with soluble A-beta to modulate chemokine/cytokine enriched AD microenvironment. Freshly isolated neutrophil labeled with green membrane dye plated on angular chamber and incubated under microscope to observe their movement and accumulation in central chamber. We observed the significant neutrophil recruitment modulated by activated microglia with A-beta. The neutrophil recruitment was assessed by measuring the speed, persistence of recruitment, and counting the number of accumulated neutrophils. We found that microglia-derived IL6, IL8, CCL2, CCL3/4, and CCL5 by A-beta stimulation are effective for recruiting neutrophils in the recapitulated AD environment and observed the significantly reduced neutrophil recruitment by neutralizing antibodies for IL6, IL8, and CCL2. This discovery provides new insights to understand the interactions between central and peripheral immune cells in human AD brains and strategies to modulate AD neuroinflammation for the therapeutic purpose.

Glioma cells in situ are surrounded by microglia, suggesting the potential of glioma-microglia interactions to produce various outcomes. Here, we reconstituted a microenvironment of boosted brain tumor invasion by inducing glioma migration by using EGF, a chemoattractant for glioma, and promoting the migration via the incorporation with microglia. We plated both glioma and microglia on the outer chamber and EGF in the central chamber, which are connected through migration channels (50×10×500 μm in width, height, and length) forming gradients of chemokines for glioma. We found PAI-1 is abundantly expressed on most glioma lines. Next, we culture the U87 glioma line, which has produced PAI-1, to investigate the hypothesis that glioma-secreted PAI-1 interacts with microglia to affect glioma migration. In co-culture with microglia in a microfluidic device, the invasiveness of PAI-1 expressing U87 cell lines was increased. Cytokine array analyses were then undertaken and they revealed that interleukin (IL)-6 was consistently increased in the co-culture. Recombinant IL-6 enhanced the invasiveness of glioma cells when these were cultured alone, whereas a neutralizing antibody to IL-6 attenuated the microglia-stimulated glioma invasiveness. This study has uncovered a mechanism by which glioma cells exploit microglia for increased invasiveness. Specifically, glioma-derived PAI-1 acts upon microglia, which then produces IL-6 to stimulate gliomas. The PAI-1/IL-6 loop is a potential therapeutic target for the currently incurable malignant gliomas.

### BRIEF DESCRIPTION OF THE DRAWINGS

FIG. 1A. Schematics describe blood and brain parenchyma regions in a human AD brain. Overexpressed neurotoxic A-beta (gray) peptides activate microglia (green) in AD brains (left) and induce inflammatory mediators that act as a compass for neutrophil (red) chemotaxis (right).

FIG. 1B. Our model mimics AD microenvironments. Microglia are loaded in a central microchamber with soluble A-beta (left). Activated microglia induce the gradients of chemokines and consequently neutrophils in an angular microchamber migrate and accumulate in the central chamber (right).

FIG. 2. A top view of one embodiment of a microfluidic device.

FIG. 3. A lateral view of one embodiment of a microfluidic device.

FIG. 4. A lateral view of one embodiment of a microfluidic device.

FIG. 5. A profile view of one embodiment of a microfluidic device.

FIG. 6. An exploded profile view of one embodiment of a microfluidic device.

FIG. 7. An embodiment of an array of microfluidic devices.

FIG. 8. An embodiment of an array of microfluidic devices.

FIG. 9A. Activation of microglial inflammation with A-beta.

FIG. 9B. Chart of chemokine release

FIG. 9C. Photos of microglia morphogenesis

FIG. 9D. Chart of marker expression.

FIG. 9E. Chart of marker expression.

FIG. 9F. CD68 immunofluorescence stain on A-beta stimulated microglial cells. Membrane stained microglial cells (red) immunostained with anti-CD68 to observe activation of microglial cells with A-beta.

FIG. 10A. Design and experimental validation of a microfluidic chemotactic model for neutrophil migration. Neutrophils (green) instantly migrated along microchannels designed for neutrophils with a gradient of fMLP (10 nM) and accumulated in a central chamber, the source of fMLP.

FIG. 10B. Counting the accumulated neutrophils in the central chamber for 6 hours shows persistent chemotactic behavior of neutrophils responding to fMLP (10 nM, black), fMLP (1 nM, dark gray), A-beta (22 nM, gray), and a negative control (media, light gray).

FIG. 10C. Time-lapse images show a simultaneous bi-directional migration of neurophils under the fMLP gradient on a single cellular level. The red arrows indicate persistent forward migration of neutrophils along the gradient of the chemoattractant and green arrows indicate the backward migration against the gradient.

FIG. 10D. Measuring the migration persistence at 1 hour shows the dominancy of the forward migration of neutrophils for fMLP. Combined data from n=5 independent experiments. Scale bars: (10A) 500 μm, (10C) 50 μm. Data represented as mean±SEM.

FIG. 10E. Design and experimental validation of a microfluidic chemotactic model for neutrophil migration.

FIG. 10F. Schematic representation for the definition of the recruitment index, R.I. shown in FIG. 10F. Neutrophil recruitment was quantified by comparing the fraction of cells inside the central chamber (CC) to the total number of cells in the corresponding angular chamber device (AC). The R.I. is calculated as R.I.=(CC_{Day i}−CC_{Day 0})/AC_{Day i}, where Day i is the number of cells in the central chamber and AD_{Day i} is the number of cells in angular chamber. The recruitment index, R.I., is obtained by normalizing with the subtraction at ‘CC_{Day 0}’ cell numbers.

FIG. 10G. Migratory speed of neutrophils migrating towards fMLP in migration channels.

FIG. 10H. Polarized neutrophil observed in co-culture condition of microglial cells stimulated with A-beta. The formation of polarized circular lamellipodia of neutrophil in co-culture condition of microglial cells (bottom) and neutrophils incubated with 100 nM fMLP (top) imaged by microscopy. Images are representative of multiple cells from four independent experiments. Scale bars, 100 μm (left column) 20 μm (right column).

FIG. 11A. Experimental validation of human neutrophil recruitment by microglia in AD.

FIG. 11B. Experimental validation of human neutrophil recruitment by microglia in AD.

FIG. 11C. Experimental validation of human neutrophil recruitment by microglia in AD.

FIG. 11D. Experimental validation of human neutrophil recruitment by microglia in AD.

FIG. 11E. Experimental validation of human neutrophil recruitment by microglia in AD.

FIG. 12A. Identification of soluble factors mediating crosstalk between microglia and neutrophils.

FIG. 12B. Identification of soluble factors mediating crosstalk between microglia and neutrophils.

FIG. 13. Design and experimental validation of a microfluidic chemotactic model for brain tumor cells.

FIG. 14. Experimental validation of co-migratory behavior of brain tumors-microglial cells.

FIG. 15. Identified soluble factors mediating crosstalk between brain tumors and microglia.

FIG. 16. 3D reconstructed human neuron packed in a gel-filled (Matrigel™) central compartment after 1 week seeding.

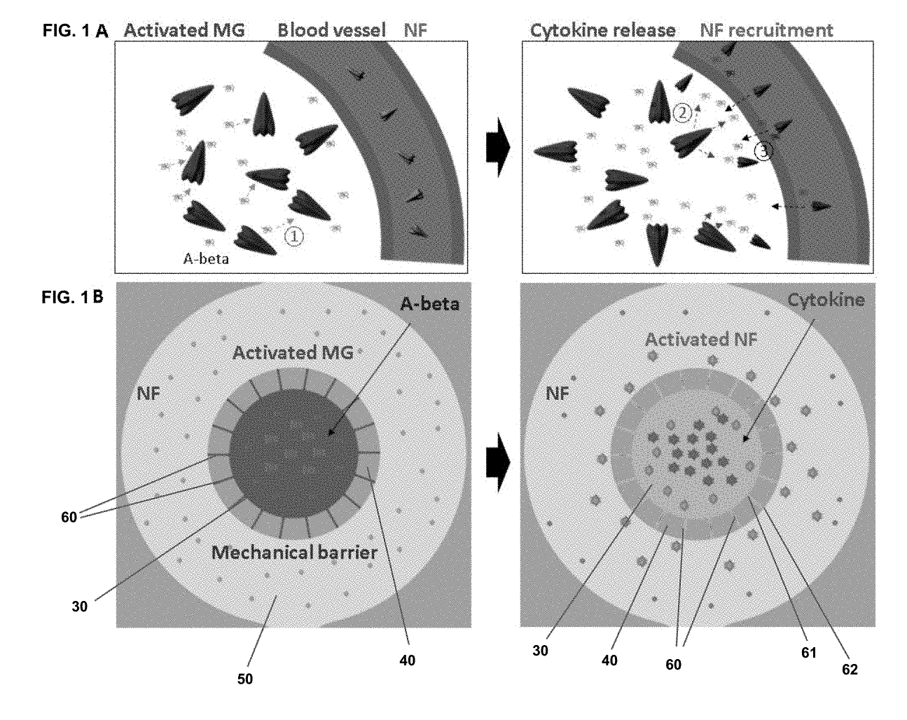

### DETAILED DESCRIPTION AND BEST MODE OF IMPLEMENTATION

The present invention now will be described more fully hereinafter in the following detailed description of the invention, in which some, but not all embodiments of the invention are described. Indeed, this invention may be embodied in many different forms and should not be construed as limited to the embodiments set forth herein; rather, these embodiments are provided so that this disclosure will satisfy applicable legal requirements.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The accumulation of immune cells in brain parenchyma is a critical step in the progression of neuroinflammatory diseases, including Alzheimer's disease (AD). The accumulation mechanisms of resident immune cells in the brain, microglia, are well studied in the context of AD pathogenesis, however, the mechanisms of peripheral immune cells are not clear, yet. Here, we reconstituted an AD microenvironment: induction of neuroinflammation by stimulating human microglia with amyloid-beta (A-beta), a signature molecule in AD, and employed this environment to investigate the recruitment mechanism of human peripheral immune cells—neutrophil—in the context of the innate-peripheral immunity crosstalk between peripheral immune system and microglia in the brain. We observed the significant neutrophil recruitment modulated by activated microglia with A-beta. The neutrophil recruitment was assessed by measuring the speed, persistence of recruitment, and counting the number of accumulated neutrophils. We found that microglia-derived IL6, IL8, CCL2, CCL3/4, and CCL5 by A-beta stimulation are effective for recruiting neutrophils in the recapitulated AD environment and observed the significantly reduced neutrophil recruitment by neutralizing antibodies for IL6, IL8, and CCL2. This discovery provides new insights to understand the central and peripheral immune cell interactions in human AD brains and strategies to modulate AD neuroinflammation for the therapeutic purpose.

The present invention includes a method for simulating a neuroinflammatory response in brains comprising the steps of:

(a) providing a microfluidic device 10 for regulating microgliosis comprising:
1. a base 20;
2. a central chamber 30 located on the base 20;
3. a size-exclusive barrier 40 surrounding the central chamber 30;
4. an annular chamber 50 outside the barrier 40;
5. a plurality of selective cellular migration channels 60 connecting the annular chamber 50 to the central chamber 30;
6. a central reservoir 80 in fluid communication with the central chamber 30; and
7. two or more side reservoirs 90 in fluid communication with the annular chamber 50;

(b) providing a camera to record the microfluidic device 10 and cells;

(c) culturing a plurality of microglial cells within the central 30 or annular 50 chambers;

(d) culturing a plurality of a second cell type within the annular 50 or central 30 chambers;

(e) adding a chemoattractant or stimulating cues inducing chemoattractants to the central chamber 30; and

(f) recording the microfluidic device 10 for a period of time to record the morphogenesis of activated cells in the annular chamber 50 and migration across the barrier 40 through the plurality of selective cellular migration channels 60.

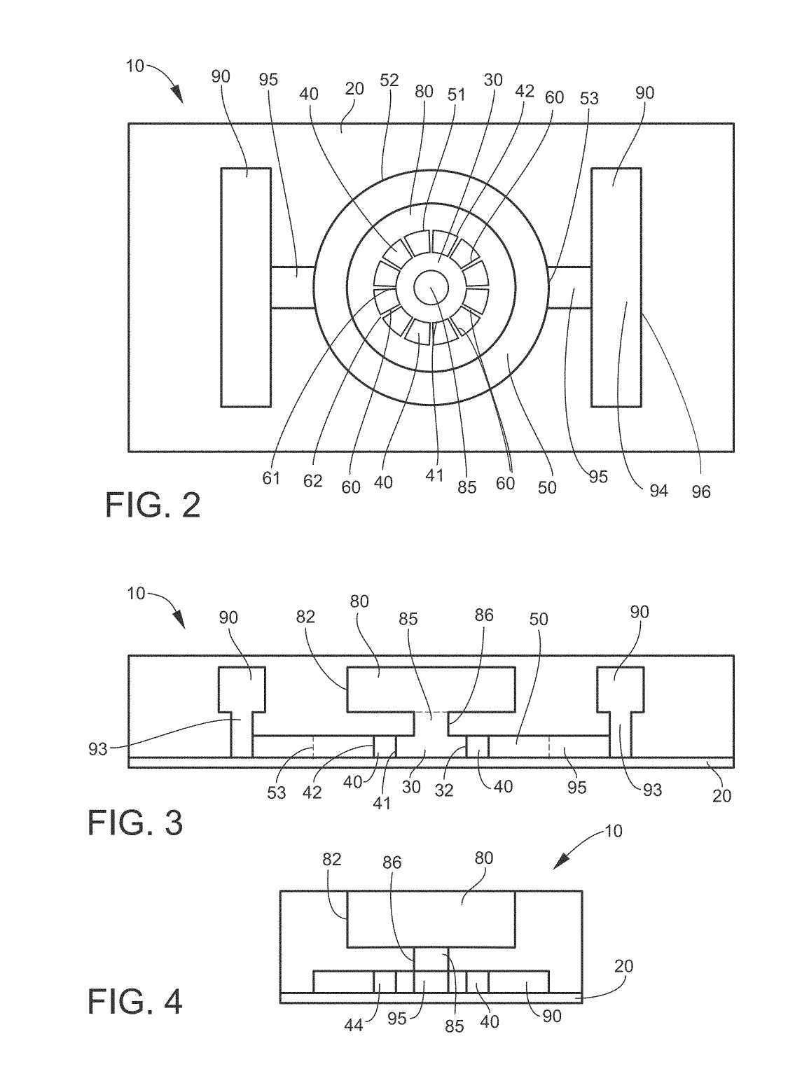

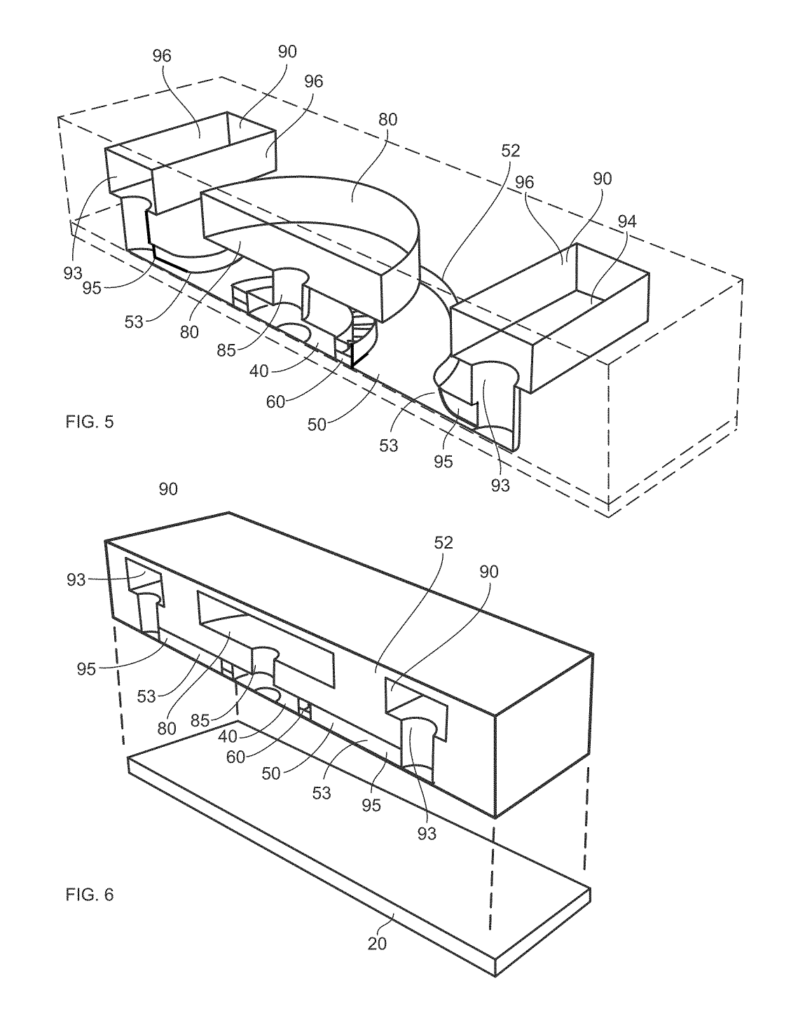

FIGS. 2-6 illustrate several embodiments of a microfluidic device 10 and its various components. A microfluidic device 10 includes a base 20 and a central chamber 30 secured to the base 20. The central chamber 30 has an outer wall 32 which defines the outer perimeter of the central chamber. A barrier 40 surrounds the central chamber 30 and the barrier 40 includes a frame 44, an inner wall 41 which defines the inner perimeter of the frame and an outer wall 42 which defines the outer perimeter of the frame. A plurality of migration channels 60 (i.e. a fluid-flow path) pass through the frame 44 of the barrier 40. The migration channels 60 include an inner channel opening 61 located on the inner wall 41 of the barrier and outer channel opening 62 located on the outer wall 42 of the barrier. (A more thorough description of the migration channels 60 is provided below.) An annular chamber 50 surrounds the barrier 40 and includes an inner wall 51 which defines the inner perimeter of the annular chamber and an outer wall 52 which defines the outer perimeter of the annular chamber. The microfluidic device 10 further includes a central reservoir 80 in fluid communication with the central chamber 30. The central reservoir 80 includes an outer wall 82 which to find perimeter of the central reservoir. As shown in FIGS. 2 through 6, the central reservoir 80 may be located above the central chamber and connected to the central chamber through a central channel 85. The central channel 85 is defined by an outer wall 86. In one embodiment, the central channel 85 is also a migration channel. The microfluidic device may also include one or more side reservoirs 90 which are in fluid communication with the annular chamber 50. Looking to FIGS. 2 through 6, there is illustrated an embodiment of a pair of side reservoirs 90 which are defined by an outer wall 92 which may be comprised of a plurality of sides 96 and a bottom 94. An opening 93 is located on the bottom 94 which leads to a side channel 95 which is in fluid communication with the annular chamber 50 through an opening 53 in the outer wall 52.

Neuroinflammation or neuroinflammatory response is an organism's biological response to harmful stimuli such as pathogens, irritants or damaged cells within nervous tissue. In the central nervous system (CNS), glial cells surround neurons and provide support for and insulation between them. The major distinction is that unlike neurons, glia do not participate directly in synaptic interactions and electrical signaling, although their supportive functions help define synaptic contacts and maintain the signaling abilities of neurons. Glia are more numerous than nerve cells in the brain, outnumbering them by a ratio of approximately 10 to 1. Although glial cells also have complex processes extending from their cell bodies, they are generally smaller than neurons, and they lack axons and dendrites. Glial cells come in a variety of forms including oligodendrocytes, astrocytes, ependymal cells, Schwann cells, microglia, and satellite cells. Microglia are located throughout the CNS and account for 10-15% of the cells found within the brain. Microglia are the resident macrophage cells and they act as the first and primary form of active immune defense of the CNS. Microglia are key cells in overall brain maintenance, thus they are continually hunting through the CNS for infectious agents, plaques and damaged neurons and synapses to dispose of them. Microglia's role in neurodegeneration is still under study. Neuroinflammation often involves microglia responses that may produce neurodegenerative symptoms including proinflammation, dystrophic nitrite growth, and excessive tau phosphorylation. A long-term, sustained response from microglia is known to enhance and expand the effects of the damage/injury which originally activated the glial response. As the microglia remain activated for an extended period, the production of mediators is sustained longer than usual and this increase in mediators contributes to neuronal damage, synaptic impairment, memory loss.

Gliosis is a nonspecific reactive change of glial cells in response to damage to the central nervous system (CNS). In most cases, gliosis involves the proliferation or hypertrophy of several different types of glial cells, including astrocytes, microglia, and oligodendrocytes. In its most extreme form, the proliferation associated with gliosis leads to the formation of a glial scar. Microgliosis is a reaction of CNS microglia to pathogenic insults. Put another way, microgliosis is an accumulation of microglial cells as a reaction to injury to the parenchyma of the central nervous system, a characteristic of nonsuppurative encephalomyelitis. One of the characteristic features of microgliosis is an increase in the number of activated microglia at the site of lesion.

One aspect of the present invention is directed to a device or system, particularly a multi-path microfluidic device. “Microfluidic device 10,” as used herein, refers to a device, apparatus or system including at least one migration channel 60 having a dimension (e.g., height, length or depth) between 1 and 1000 microns (μm). “Fluid-flow path,” “fluid path” or “flow path” as used herein, refer to any migration channel 60, tube, region, space or pathway or portion thereof through which a fluid, including a liquid or a gas, may pass. A “fluid flow passageway” includes a portion of a migration channel 60. The device also includes an optically transparent material that is coupled to the substrate. The device further includes a barrier 40 that contacts (for example, by filling the space between) the substrate and the optically transparent material, generally within a defined region of the device. As used herein, the phrase “in three-dimensional space” refers to having the quality of being three-dimensional, as these dimensions exist in three-dimensional circular geometry. However, non-circular spaces and shapes are also included in the invention. The substrate is generally a solid material, such as poly-dimethyl siloxane (PDMS), formed by a soft lithography process, in which the fluid-flow paths are channels in the substrate. Where two migration channels 60 are present in the same substrate, these migration channels are substantially parallel along at least part of their lengths. Each migration channel 60 may contain a fluid inlet (enter channel opening 61) and a fluid outlet (outer channel opening 62). Fluid inlets are holes, channels or other means for a fluid such as cell culture media to be conducted from outside the device into the fluid-flow path. Fluid outlets are also holes, channels or other means for a fluid such as conditioned or waste cell culture media to be conducted away from the device. One of skill in the art will recognize that other materials used in microfabrication or microfluidics fields are useful in producing the substrate of the invention. For example, a substrate may be formed from or contain silicon, glass, quartz, or plastic (e.g., any synthetic or semi-synthetic polymer having the necessary structural attributes to function as a substrate). Useful plastics are known to those skilled in the art. The fluid-flow paths can be varied in any dimension (e.g., length, width or depth) to produce a desired flow resistance. In various embodiments of the instant invention, the migration channels 60 of the microfluidic device 10 have a width in the range of 2 to 20 μm, 2 to 15 μm, 3 to 15 μm, 5 to 12 μm or 5 to 10 μm. In various embodiments of the instant invention, the migration channels 60 of the microfluidic device 10 have a width of 1, 2, 3, 4, 5, 6, 7, 8, 9, 10, 11, 12, 13, 14, 15, 16, 17, 18, 19 or 20 μm. In various embodiments of the instant invention, the migration channels 60 of the microfluidic device 10 have a height in the range of 2 to 20 μm, 2 to 15 μm, 3 to 15 μm, 5 to 12 μm or 5 to 10 μm. In various embodiments of the instant invention, the migration channels 60 of the microfluidic device 10 have a height of 1, 2, 3, 4, 5, 6, 7, 8, 9, 10, 11, 12, 13, 14, 15, 16, 17, 18, 19 or 20 μm. In various embodiments of the instant invention, the migration channels 60 of the microfluidic device 10 have a length in the range of 100 to 900 μm, 50 to 1000 μm, 200 to 800 μm, 300 to 700 μm, 400 to 600 μm, or 500 μm.

The barrier 40 contained within the device separates the central chamber 30 and annular chamber 50 connected through migration channels 60 providing a multifunctional support upon which cells can migrate, proliferate, or differentiate, depending upon physiological conditions provided in the device. Prokaryotic cells including bacteria are included, as are eukaryotic cells. More than one cell type may be introduced into the barrier 40, either concurrently or consecutively. Generally, the barrier 40 contains a solid or semi-solid biological or biocompatible material (or biomaterial), often which is in the form of a polymer. Described herein are cell assay devices that can be used to screen for agents that have an effect on living cells. For example, the device can be used to screen for anti-angiogenesis agents, anti-metastasis agents, wound healing agents and tissue engineering agents. In one aspect, the device allows blood vessels to be seen from the side, not as holes but as cones, enabling a more detailed analysis of the effects of agents (e.g., drugs) on the growth of cells (e.g., invasion of cancer cells).

In particular aspects, the device is a 3D Microfluidic Device that can be used in a high throughput manner for investigations of multi-cellular interactions (e.g., high throughput AD drug screening). The device can be used for drug discovery and personalized medicine. For example, the device can be used as an in-vitro test for new drugs that may help stop the spread of cancer in the human body in a research setting, and to match the best drug to AD patients in a hospital setting. This device is an improvement over current devices in that it allows easy imaging and quantitative, not just qualitative, results. In one aspect, the device comprises a substrate comprised of an optically transparent material and (i) one or more fluid chambers; (ii) one or more fluid chamber holes; (iii) one or more fluid chamber outlets; (iii) one or more gel cage regions;

Here, we present a microfluidic device 10 and for the modeling of neutrophil migration in vitro via soluble A-beta activated microglia. Our device offers key advantages by enabling all the following: (1) high temporal and spatial resolution of neutrophil migration, (2) the ability to perform parametric studies in a tightly controlled and high throughput screening test and (3) physiologically relevant environment by modulating AD environment with two separate chambers. We employ our model to demonstrate the effect of soluble A-beta stimulation on microglial cells and their chemokine secretion, and the positive correlation with neutrophil recruitment. We demonstrated high spatiotemporal visualization of the neutrophil migration by quantifying their persistent movement with various chemokine stimulation, and observed that IL6/IL8/CCL2/CCL3/CCL5 are crucial factors for the recruitment of neutrophil by validating with neutralization of antibody. Findings from our device result in a deeper understanding of neutrophil movement mechanisms and demonstrate our assay's potential to be employed for the discovery of new factors that could inhibit this crucial step in inflammation. Taken together, these studies suggest that microglia in the brain innate immune cells have a critical role in recruiting peripheral immune cells in the development or progression of AD.

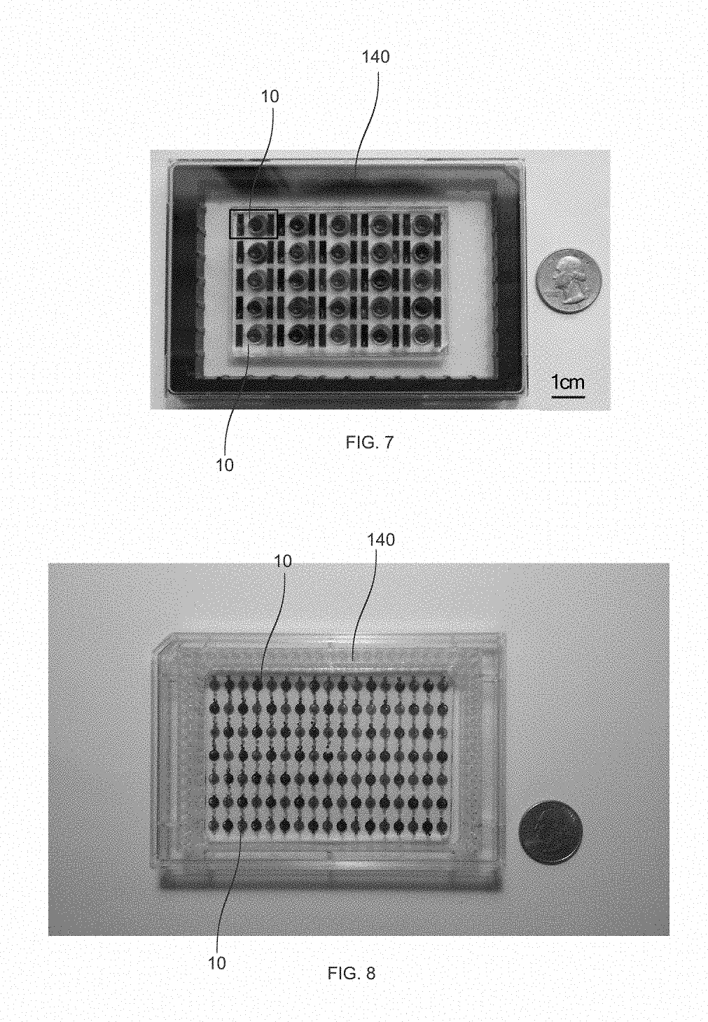

Looking now to FIGS. 7 and 8, there is illustrated embodiments of an array of microfluidic devices 10 on a well plate 140. Each well plate 140 contains a plurality of microfluidic devices 10 wherein each microfluidic device can test identical cells, non-identical cells or a combination thereof.

In Vitro Modulation of AD Microenvironment with Mechanical Barrier in Microfluidic Device

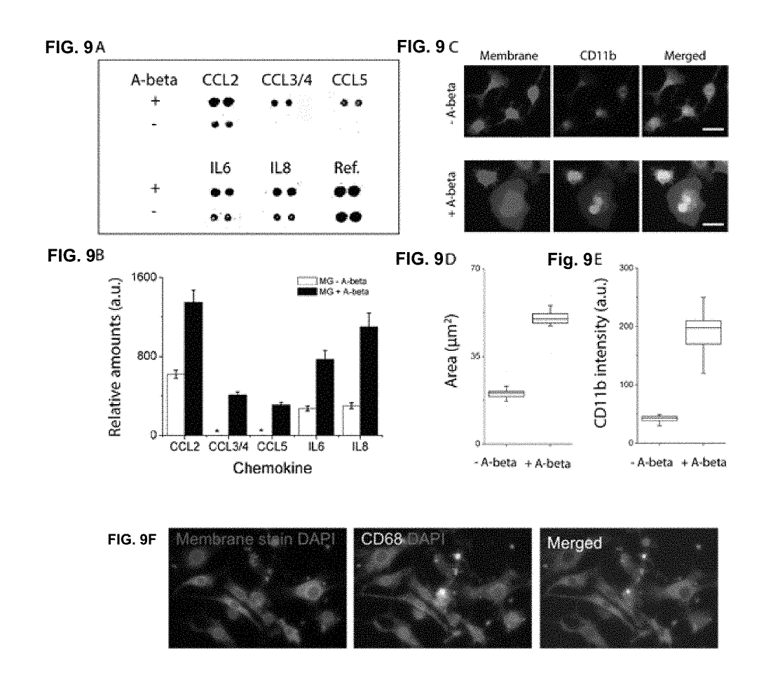

Microglial cells are resident brain immune cells and stimulated by soluble A-beta42, which is a signature of human AD neurotoxic peptide. Based on our previous observation of neutrophil accumulation on A-beta plaques in in vivo, we hypothesize that A-beta activated microglial cells has a critical role in recruiting neutrophil. Stimulated microglial cells can release various types of chemokines and/or cytokines to increase inflammatory behavior or to clear A-beta in inflamed region. Furthermore, those secreted chemokines and/or cytokines can recruit other cell types including peripheral immune cells (FIG. 9A). To modulate this whole process, we established an in vitro AD brain-blood model (ADBBM) using microglia and neutrophil to mimic the brain microenvironment. To accommodate the different cells separately in a single microfluidic platform, we employed mechanically separated two chambers, which are connected through neutrophil specific migration channels (10×5×500 μm in width, height, and length) forming gradients of chemokines and/or cytokines (FIG. 9B).

Compared to a parallel configuration having equal surface areas for cell loading and cell collection, the asymmetric configuration of the current microfluidic device 10 has a small central cell collecting compartment (central chamber 30), which is smaller than a large annular cell loading compartment (annular chamber 50) by a factor of 3, 4, 5, 6, 7, 8, 9, 10 or greater. We tested the stability of chemical gradients within the device channels 60, 85, 95, and found that they remained constant for ˜72 h. This enhances the gradient formation and detection of recruitment. The long and thin migration channels 60 can block spontaneous entrance of inactivated neutrophil through mechanical constraints. Using this platform, we could culture microglial cells for extended periods to accumulate enough soluble factors and could observe neutrophil signature movement at single cell resolution, in real-time. Red membrane dye stained microglial cells plated first in central chamber 30 and incubated for 3 days with soluble A-beta to modulate chemokine/cytokine enriched AD microenvironment. Freshly isolated neutrophil labeled with green membrane dye plated on annular chamber 50 and incubated under microscope to observe their movement and accumulation in central chamber 30. Precise numbers of neutrophils are reproducibly loaded and uniformly distributed in annular chamber 50 and this design allows us to evaluate the response of neutrophil to various types of chemokines and/or cytokines. We tested 200 independent conditions at the same time, by using real time imaging of process in an array of twenty-five formatted on a single well plate 140 or an array of one-hundred nineteen formatted single well plate 140 (FIG. 7 and FIG. 8).

### Soluble A-Beta Induce Chemokine Secretion and Phenotype, Morphology Changes From Activated Microglia

To characterize the phenotype changes in microglia induced by soluble A-beta, individual microglial cells were monitored for three days using time-lapse imaging microscopy. During the first 24 hours of culturing microglia in the central chamber 30, the cells showed widely branched filopodia in all directions, morphology typically associated with ‘resting’ microglia (FIG. 9C top, −A-beta). Morphological changes could be observed at 48 hrs following cell seeding (FIG. 9C bottom, +A-beta). As determined by image analysis, microglial cells became large and switched into more amoeboid type as the length and area of microglial somata (cell bodies) increased in all direction (FIG. 9C). And filopodia, which is observed in resting mode microglia, could not be observed in A-beta stimulated microglia. Furthermore, exposure of microglia to soluble A-beta induced up-regulation of microglial activation markers such as CD11b (FIGS. 9D and 9E) and CD68 (FIG. 9F). FIG. 9F shows a CD68 immunofluorescence stain on A-beta stimulated microglial cells. Membrane stained microglial cells (red) immunostained with anti-CD68 to observe activation of microglial cells with A-beta.

As microglia are the predominant resident immune cells of the brain, we next determined the effects of the soluble A-beta AD microenvironment on the inflammatory activity of microglia. Exposure to soluble A-beta was associated with significant increases in chemokine release (FIG. 9B). CCL2, IL-6 and IL-8 concentrations were increased by 1.4-, 1.9- and 2.3-fold, respectively, and could be observed new chemokine release of CCL3/4, CCL5 compared to microglia without A-beta alone. Secretion of anti-inflammatory markers, such as IL-1RA, IL-4, IL-10 and TGF-β was very low or no detectable. These data suggest that microglia elicited broad chemokine signaling in our co-culture model of AD.

Platform Validation with Standard Chemoattractant, fMLP, for Neutrophil

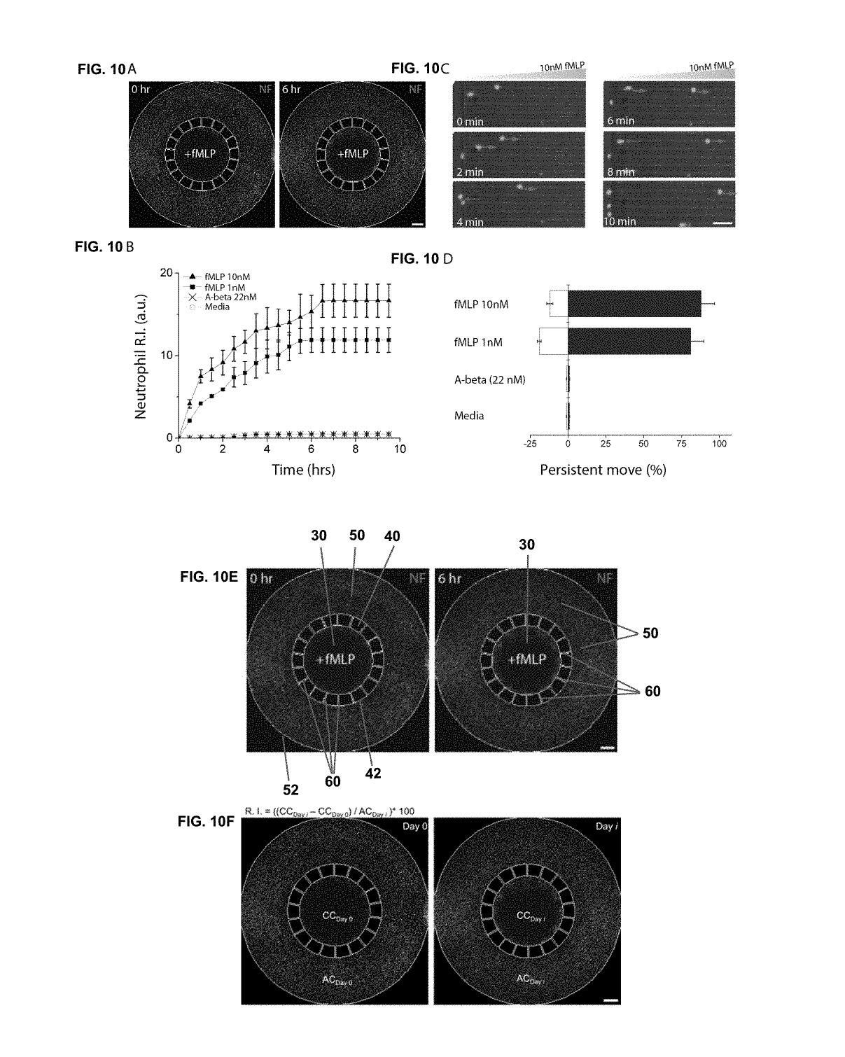

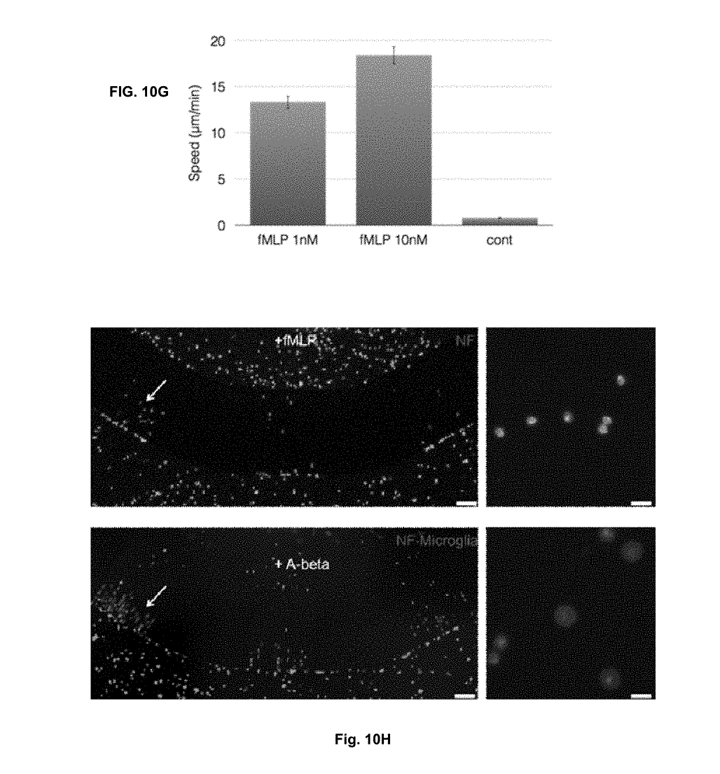

We validated the assay by measuring the migration and accumulation of neutrophils toward a panel of known chemoattractant. To compare the effect of fMLP in various concentrations on neutrophil recruitment, we defined a neutrophil recruitment index, R.I. (FIG. 10B, FIG. 10F), representing the fraction of neutrophil recruited to the central chamber after loading fMLP in the central chamber. We compared the R.I. values under various soluble factors, (fMLP, A-beta, and media) (FIG. 10B) and could observe predominant neutrophil (green) recruitment with N-formyl-methionine-leucine-phenylalanine (fMLP) (10 nM) in central chamber 30 after 6 hours (FIG. 10A). A negligible number of cells entered central chamber in the absence of a chemoattractant by proving that the mechanical barrier 40 can exclude the spontaneous entrance of neutrophil. Neutrophils showed a strong and rapid response, reaching a plateau within the first 6 hours and the magnitude of cell recruitment was 1.6 times higher with 10 nM fMLP compared to 1 nM fMLP (FIG. 10B). Neutrophils move through migration channels 60 at 18±5 μm/min and could reach central chamber in less than 30 min (FIG. 10G). We compared the migratory response of human neutrophils with various stimulators. We found that a total of 88.5±3.8% neutrophils migrated towards 10 nM fMLP and 11.5±1.7% migrated back from it, whereas in 1 nM fMLP chemoattractant was noted in 82.2±3.4% and migrated back in 17.8±2.8% of the neutrophils showing that neutrophil behavior has a dependency on chemoattractant concentrations (FIG. 10C, 10D). Concentration-dependent changes in migration patterns in response to fMLP was primarily affect the migrating faction while there was no significant effect on persistent directionality. Increasing fMLP concentrations from 10 nM to 1 μM resulted in a 63% decrease in the total fraction of migrating cells (FIG. 10H).

### Validation the Dependency of Neutrophil Migratory Signatures on A-Beta Activated Microglia

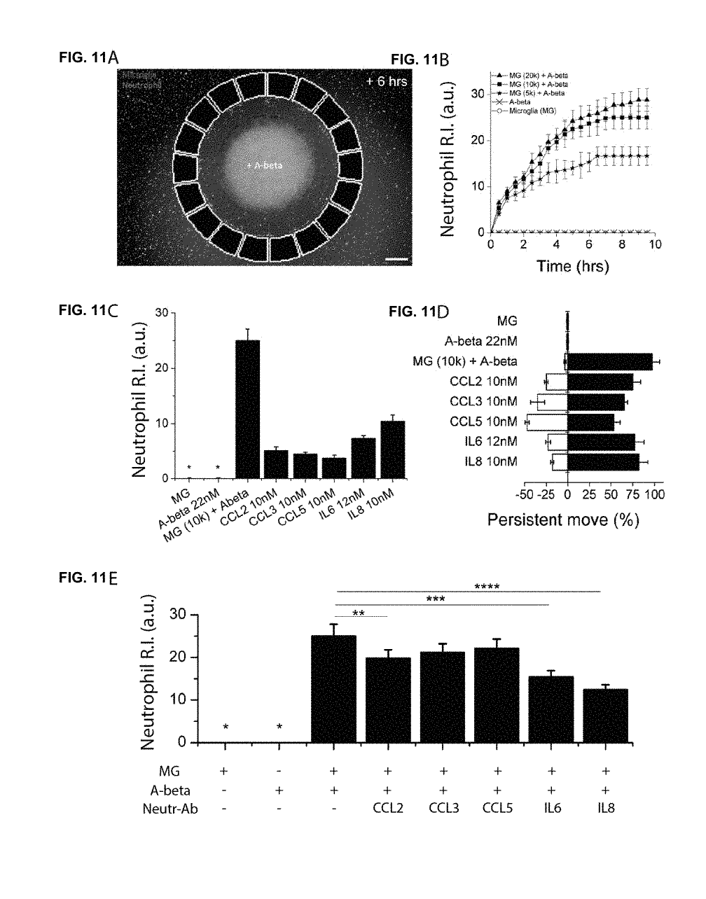

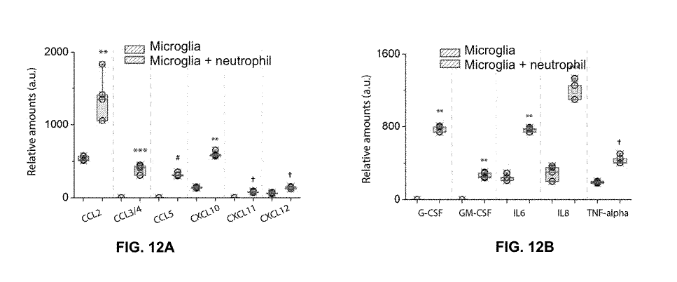

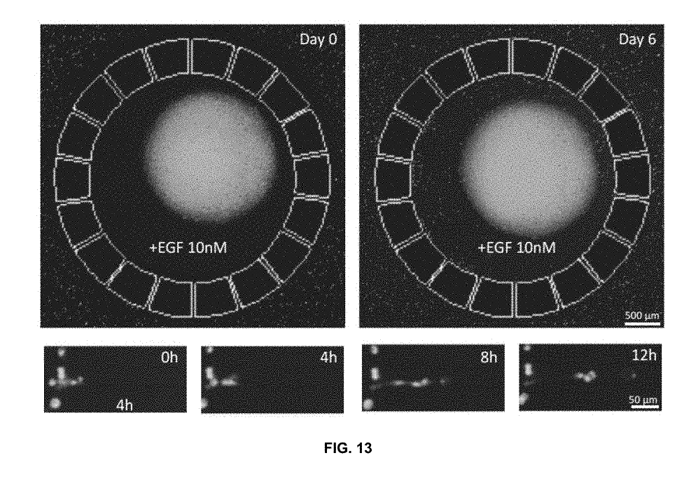

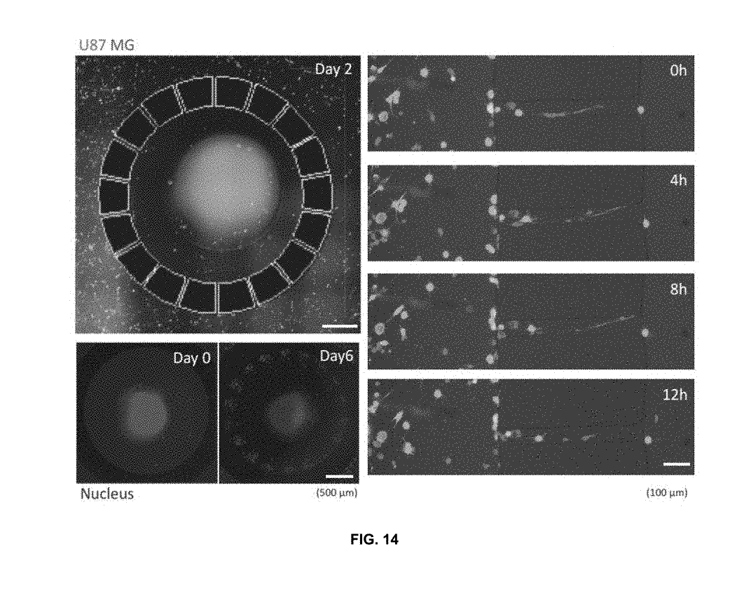

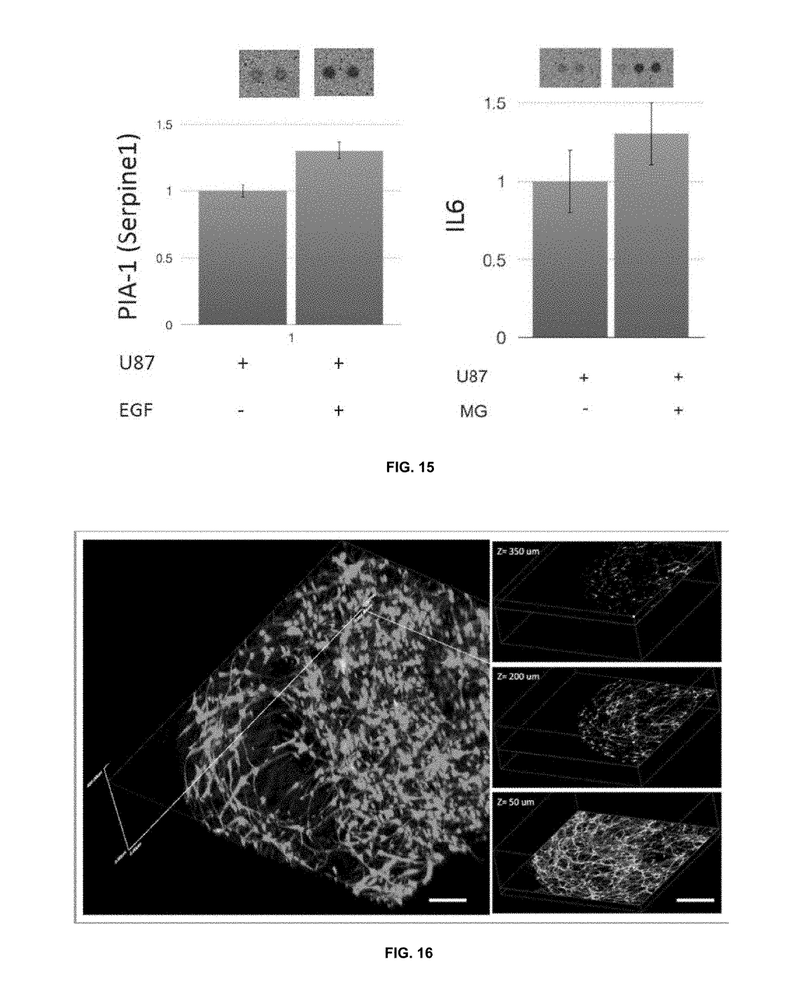

To validate the dependency of neutrophil migration toward soluble A-beta activated microglia, microglial cells plated on central chamber 30 with various cell numbers (5,000, 10,000, 20,000 cells). Microglial cells are pre-labeled with membrane dye according to manufacturer's protocol to observe their morphology change. After 24 hour incubation of microglia alone, prepared soluble A-beta (2.2 uM) with reduced 1% FBS Prigrowlll media were changed to incubate further for 48 hours. After 48 hours, freshly isolated neutrophil labeled with green membrane dye plated on annular chamber 50 and incubated on microscopic real time imaging chamber. Neutrophil (green) migration could be observed toward soluble A-beta activated microglia (red) cultured central chamber 30 after 6 hours (FIG. 11A). 2.4-, 1.2-times higher neutrophil migration could be observed in central chamber 30 in response to 10,000 and 20,000 cultured microglia in central chamber 30 by showing the dependency of neutrophil recruitment in cell numbers. Negligible neutrophil migration could be observed in microglial cell alone without A-beta, medium and A-beta alone (FIG. 11B). By utilizing high-resolution and spatiotemporal controlled microscopic imaging method, we also can quantify the persistent movement of microglial cells in various stimulations. With co-culture of soluble A-beta activated microglial cells, neutrophil can migrate with high persistent about 92% (FIG. 11C) compare to fMLP alone in FIG. 10B. Co-culture with A-beta activated microglial cell induced high-ratio persistent movement of neutrophil regardless of cell numbers in central chamber. We hypothesize the main reason of this high ratio of persistent movement can be rely on the presence of several chemokine in central chamber. To validate the crucial recruitment factor of neutrophil, we incubated one each chemokine factors which are detected in FIG. 9B. We identified five cytokines at detectable levels: CCL2, CCL3/4, CCL5, IL-6 and IL-8 while the remaining cytokines were either below, or just at the threshold of detectability. Arrows indicate saturated measurement of cytokines relative to the standard curve. We measured neutrophil recruitment under gradients of soluble A-beta activated microglia in addition to a mixture of neutralizing antibodies against CCL2, CCL3/4, CCL5, IL6 and IL8 and observed reduced activity at CCL2, IL6 and IL8 (FIG. 11D).

The microglial cells used in the instant invention are selected from the group including human microglial cells, non-human microglial cells, or a combination thereof. Primary or iPSC-derived human microglial cells may be used in the instant invention. The microglial cells used in the instant invention may come from a single individual or from two or more individuals. The microglial cells used in the instant invention may come from a single individual having a neurodegenerative disorder, from two or more individuals with neurodegenerative disorders, from a single individual with no neurodegenerative disorder, from two or more individuals with no neurodegenerative disorders, or a combination thereof. The microglial cells may be microglial cells activated by soluble cues which are isolated from spontaneously activated microglia by culturing media. The non-activated microglia and activated microglial cells may be separated in a regulated manner based on microglia motility in response to chemoattractants.

The second cell type used in the instant invention is selected from the group including: peripheral immune cells, neurons, astrocytes, oligodendrocytes, endothelia, pericytes, tumor cells or a combination thereof. The peripheral immune cells used in the instant invention are selected from the group including: leukocytes (i.e. neutrophils, monocytes, macrophages, etc.) adaptive immune cells (i.e. T cells, B cells, etc.) or a combination thereof.

The stimulating cues used in the instant invention are selected from the group including: Amyloid beta, LPS, CCL2, ATP, EGF, neuroinflammatory soluble cues, conditioned media from other neurodegenerative cells or a combination thereof. The chemoattractant used in the instant invention is selected from the group including: Amyloid beta, LPS, CCL2, ATP, or a combination thereof. In various embodiments, the chemoattractant may further include one or more cytokines released from either microglia or the second type of cells by the stimulating cues and including: IL-1ra, IL-6, IL-8, MCP-1, MIP-1b, or any combination thereof.

In one embodiment of the instant invention, at least a portion of the microfluidic device 10 is coated with a biocompatible agent (i.e. Collagen, Matrigel™, Poly-D-lysine, Poly-L-lysine, fibronectin) which facilitates cellular attachment to and migration on the device.

Provided herein are microfluidic devices 10 that can be used as a 3D bioassay or 3D culturing environment, e.g., for drug screening, personalized medicine, tissue engineering, wound healing, and other applications. The device has an array of side channels connecting central and annular chambers wherein the channels and the central chamber can be filled with a biologically relevant gel, such as collagen and/or Matrigel™. The gel is either naturally isolated or synthesized and is selected from the group including: Matrigel™, Collagen, Gelatin, Hyaluronic Acid, Alginate, Polyethylene glycol (PEG), or any combination thereof. As shown herein, when the device is plated with cells such as neuroprogenitor cells, neurons will grow in the gel, which is thick enough for the cells to grow in three dimensions. Other channels, e.g., fluid channels, allow drugs or biological material to be exposed to the 2D cell growth in angular chamber. Cells, such as neurons and microglia, can be cultured in the central compartment filled with gels to mimic further physiological relevant environments. (FIG. 16)

We have developed a microfluidic device 10 containing two separate chambers to mimic AD brain and blood vessel environment to study the crosstalk in between the innate microglia and peripheral immune cells. We demonstrated that microglia with soluble A-beta exhibits a morphology change, expresses characteristic cellular marker upregulation and secretion of several chemokine. Moreover, we showed that the functionality of our device by showing that the quantitative measurements of neutrophil recruitment in both high spatial- and temporal-observation utilized to provide more precise measurements of migratory speed and persistent movement. The patterns of cell migration through these microchannels are distinct compare to two-dimensional flat surfaces encountered in traditional cell migration assays. The present platform can be scalable in large array form to screen multi-recruiting factors. Most importantly, this platform supports the development of a more physiologically relevant in vitro AD brain model, including the addition of other cell types such as endothelial cells, neuron or monocyte. Thus, we believe that the neutrophil-microglia microfluidic device 10 will be a useful platform for the studies such as comparative therapeutic tests with the opportunity to assess drug effects on the restriction of neuroinflammatory function.

### Cell Lines, Media and Reagents:

Immortalized microglial cells (SV40-microglia) were purchased from Abm company (ABM Inc., Montreal, Canada). The cells were plated onto T25 cell culture flasks (BD Biosciences, San Jose, Calif., USA) and maintained in Prigrow III Medium (ABM Inc) supplemented with 50 mL FBS (Life Technologies) and 5 mL Pen/Strep (Invitrogen) in a CO2 cell culture incubator. Cell culture media were changed every 3 days until cells were confluent. For neutrophil isolation, human peripheral blood samples from healthy volunteers, aged 18 years and older, were purchased from InvivoGen. Peripheral blood was drawn in 10-ml tube containing a final concentration of 5 mM EDTA (Vacutainer; Becton Dickinson). Nucleated cells were isolated using a HetaSep gradient, followed by the EasySep Human Neutrophil Enrichment Kit (STEMCELL Technologies, Vancouver, Canada) according to the manufacturer's protocol.

### Microfluidic Device Fabrication:

Negative photoresists, SU-8 50 and SU-8 100 (MicroChem, Newton, Mass., USA), were sequentially patterned using standard lithography on a 4″ silicon wafer to create a mold for cell migration channels of 10 μm in height and chemokine compartments of 100 μm in height. A mixture of a base and a curing agent with a 10:1 weight ratio (SYLGARD 184 A/B, Dow corning, Midland, Mich., USA) was poured onto the SU-8 mold and cured for one hour at room temperature under vacuum and, subsequently, cured for more than 3 hours in an oven at 80° C. The cured poly dimethyl-siloxane (PDMS) replica was peeled off from the mold and holes were punched for fluid reservoirs. Arrayed holes were also laser-cut (Zing 24, Epilog Laser, Golden, Colo., USA) into a thin PDMS membrane of 250 μm in thickness (HT 6240, Bisco Silicones, Elk Grove, Ill., USA) and an acrylic plate of 6 mm in thickness. The machined membrane and the plate were glued together using uncured PDMS and incubated at 80° C. overnight. This assembly was irreversibly bonded first to the PDMS replica using oxygen plasma at 50 mW, 5 ccm, for 30 seconds (PX-250, March Plasma Systems, Petersburg, Fla., USA), and later to a glass-bottomed Uni Well plate (MGB001-1-2-LG, Matrical Bioscience, Spokane, Wash., USA). Immediately after the bonding, 10 μL of poly (l-lysine) solution (PLL, M.W. 70,000-150,000, 1.0 mg/mL, Sigma-Aldrich Co. LLC, St. Louis, Mo., USA) was injected into each platform and incubated for 2 hours at a room temperature to promote cellular adhesion. PLL-treated surface was rinsed with autoclaved and 0.2 μm filtered water (AM9920, Life Technologies, Grand Island, N.Y., USA) and then the devices were filled with cell culture medium containing 50550 of DMEM: F-12 supplemented with 5% FBS (Invitrogen, Grand Island, N.Y., USA), 10 ng.mL⁻¹ of MCSF (AF-300-25, PeproTech Inc., Rocky Hill, N.J., USA), 25 mg.mL⁻¹ of gentamicin (G1397, Sigma-Aldrich), and 2.5 mg.mL⁻¹ of amphotericin (A2411, Sigma-Aldrich).

### Membrane Staining of Microglia and Neutrophil

Human microglial cells (ABM Inc., Montreal, Canada) were isolated initially as a free-floating population of cells from fetal brain tissue samples digested with collagenase and grown in a 10% FBS with Prigrow III media (ABM). Before the experiment, cells were washed using medium without serum and the cell membrane was labeled with red fluorescent dye (PKH26PCL, Sigma-Aldrich). Briefly, after centrifugation (400 g for 5 minutes), the cells were re-suspended in 1 mL of Diluent C (G8278, Sigma-Aldrich) and immediately mixed with 4 μL of dye solution (PKH26PCL, Sigma-Aldrich). The cell/dye mixture was incubated at room temperature for 4 minutes and periodically mixed by pipetting to achieve a bright, uniform, and reproducible labeling. After the incubation, the staining was stopped by adding an equal volume (1 mL) of 1% BSA in PBS and incubating for 1 minute to remove excess dye. Unbound dye was washed by centrifugation and suspending cells in culture medium (106 cells/ml). Ten μL of the cell suspension was injected into each platform and 100 μL of a culturing medium was added into side and central extra wells. The loaded 3D micro-device was then incubated at 37° C. supplied with 5% CO2.

### Time-Lapse Imaging:

After microglia loading, cells were recorded using time-lapse imaging using a fully automated Nikon TiE microscope with a heated incubator to 37° C. and 5% CO2 (10× magnification; Micro Device Instruments, Avon, Mass., USA). To achieve accurate cell tracking, the maximum time resolution of acquisition was 0.2 frames per second.

### Immunostaining

For immunofluorescent stain, we rinsed the cells in medium twice with DPBS. To fix, cells were incubated in fresh 4% paraformaldehyde aqueous solution (157-4, ElectronMicroscopy Sciences) for more than 15 minutes at RT followed by rinsing twice with DPBS. To permeabilize, cells were incubated in 0.1% Triton X-100 in PBST (phosphate buffered saline with 0.1% tween®20) for 15 minutes at RT. To block, cells were incubated in 3% human serum albumin for overnight in PBST at 4° C. After incubating with the primary antibody solutions for 24 hour at 4° C., the cells were washed five times. The following antibody (and dilutions) were used: anti-cd11b (1:100, Life Technologies).

### A-Beta Stimulation and Analysis of Cytokine Release

Microfluidic devices 10 were cultured for 72 hours, followed by incubation in A-beta containing medium with fetal bovine serum reduced from 10% to 1%. Microglia culture on central chamber 30 were stimulated with A-beta (Sigma Aldrich) at 2.2 uM with 2% serum for 72 hrs. The cytokine release profile was assayed with the Human Cytokine 37-membrane kit array (R&D systems) and the resulting cytokine release profiles were quantified with ImageJ analysis.

### Statistical Analysis

Data, expressed as mean±SEM, were compared using either a two-tailed Student's t-test when comparing two groups/conditions or one-way ANOVA followed by a post hoc test when comparing 3 or more groups/conditions. P value<0.05 was considered significant.

FIG. 9. Activation of microglial inflammation with A-beta: chemokine release, morphogenesis, and marker expression. (9A-9B) The expression of five chemokines among 29 membrane human cytokines measured was upregulated from stimulated microglia with A-beta at 22 nM for 24 hours (first row) compared to unstimulated microglia (second row). (9C) Membrane-staining (red) shows the discernable change in microglial shapes with A-beta stimulation from a branch-ramified shape (without A-beta, resting) to an amoeboid shape (with A-beta, activated). A microglia marker of CD11b (green) was upregulated with A-beta stimulation. Both the membrane area (9D) and the amounts of expressed CD11b (9E) increase in the activated microglia with A-beta. ndevice=3, ncell=150. Scale bars: 30 μm. All parameters are presented as mean±SEM.

FIG. 10. Design and validation of a microfluidic chemotactic model for neutrophil migration. (10A) Neutrophils (green) instantly migrated along microchannels designed for neutrophils with a gradient of fMLP (10 nM) and accumulated in a central chamber, the source of fMLP. (10C) Time-lapse images show a simultaneous bi-directional migration of neurophils under the fMLP gradient on a single cellular level. The red arrows indicate persistent forward migration of neutrophils along the gradient of the chemoattractant and green arrows indicate the backward migration against the gradient. (10B) Counting the accumulated neutrophils in the central chamber for 6 hours shows persistent chemotactic behavior of neutrophils responding to fMLP (10 nM, black), fMLP (1 nM, dark gray), A-beta (22 nM, gray), and a negative control (media, light gray). (10D) Measuring the migration persistence at 1 hour shows the dominancy of the forward migration of neutrophils for fMLP. Combined data from n=5 independent experiments. Scale bars: (10A) 500 μm, (10C) 50 μm. Data represented as mean±SEM.

FIG. 11. Co-culture of human neutrophils and microglia and reconstruction of neutrophil recruitment in AD. (11A) A fluorescent image shows recruited neutrophils (green) modulated by microglia (red) stimulated with A-beta. (11B) A plot from live-cell imaging show instant and then saturated recruitment behavior of neutrophils responding to co-culturing conditions: microglia of 20,000 cells per device (black), 10,000 cells per device (dark gray), 5,000 cells per device (gray) with A-beta at 22 nM, and negative controls (A-beta only, light gray, microglia only, white). Neutrophil chemotaxis was assessed with response to various soluble factors: A-beta at 22 nM, CCL2 at 10 nM, CCL3 at 10 nM, CCL5 at 10 nM, IL6 at 12 nM, and IL8 at 10 nM by counting the accumulated neutrophils (11C) and measuring the persistence (11D). (11E) The effectiveness of selected microglia-derived chemokines was confirmed by showing the reduced neutrophil accumulation to A-beta stimulated microglia in addition of neutralizing antibodies for individual chemokines. Statistical significance is denoted by **P<0.20, ***P<0.10, ****P<0.01 ndevice=4 (11B, 11C, 11D, 11E), nneutrophil=250. Scale bars (11A): 500 μm. All parameters are presented as mean±SEM.

One embodiment of the instant invention includes a method for simulating the neuroinflammatory response in brains comprising the steps of providing a microfluidic device for isolating a population of microglia of interest and regulating microgliosis comprising a base, a central chamber located on the base, a size-exclusive barrier surrounding the central chamber, an annular chamber outside the barrier, a plurality of selective cellular migration channels connecting the annular chamber to the central chamber, a central reservoir in fluid communication with the central chamber and side reservoirs in fluid communication with the annular chamber; providing a camera to record the microfluidic device and cells, culturing a plurality of microglial cells within the central or annular chamber, culturing a plurality of a second cell type within the annular or central chamber, adding a chemoattractant to the central chamber and recording the microfluidic device for a period of time to record the morphogenesis of activated cells in the annular chamber and migration across the barrier.

Any method described herein may incorporate any design element contained within this application and any other document/application incorporated by reference herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present invention may be embodied in other forms without departing from the spirit and the essential attributes thereof, and, accordingly, reference should be made to the appended claims, rather than to the foregoing specification, as indicating the scope of the invention. The invention illustratively disclosed herein suitably may be practiced in the absence of any element which is not specifically disclosed herein.

1. A method for simulating a neuroinflammatory response in human brains comprising the steps of:
providing a microfluidic device for regulating microgliosis comprising:
a base;
a central chamber located on the base;
an annular chamber outside the central chamber;
a plurality of size-exclusive mechanical barriers between the annular chamber and the central chamber;
a central reservoir in fluid communication with the central chamber wherein the central reservoir is supplying a culturing medium; and
two or more side reservoirs in fluid communication with the annular chamber wherein the side reservoirs are supplying a culturing medium;
culturing a plurality of microglial cells within the central or annular chamber;
co-culturing a plurality of a microglia-interacting second cell type within the annular or central chamber;
adding one or more chemoattractant or soluble factors inducing attractive cytokines to the central chamber;
isolating a subset of cells of interest from the annular to central chamber regulated by the chemoattractants or attractive cytokines in the central chamber;
providing a camera to record the microfluidic device and cells; and
recording the microfluidic device for a period of time to record the morphogenesis of activated cells in the annular chamber and migration across the barrier.

2. The method of claim 1 wherein the microglial cells are human microglial cells.

3. The method of claim 2 wherein the microglial cells are primary, iPSC-derived, immortalized human microglial cells.

4. The method of claim 2 wherein the human microglial cells are from one or more individuals having a neurodegenerative disorder.

5. The method of claim 1 wherein the second cell type is selected from the group including: peripheral immune cells, neurons, astrocytes, oligodendrocytes, endothelials, pericytes, tumor cells or a combination thereof.

6. The method of claim 5 wherein the peripheral immune cell is selected from the group including: leukocytes (i.e. neutrophils, monocytes, macrophages, etc.) adaptive immune cells (i.e. T cells, B cells, etc.) or a combination thereof.

7. The method of claim 1 wherein the chemoattractant is selected from the group including: Amyloid beta, LPS, MCP-1, PAI-1, CCL2, ATP, or a combination thereof.

8. The method of claim 1 wherein the stimulating soluble factors cueG is selected from the group including: Amyloid beta, LPS, CCL2, ATP, EGF, neuroinflammatory soluble cues, conditioned media from other neurodegenerative cells or a combination thereof.

9. The method of claim 1 wherein the chemoattractant further includes one or more cytokines released from either microglia or the second type of cells by the stimulating soluble factors and including: IL-1beta IL-1ra, IL-6, IL-8, MCP-1, MIP-1b, EGR, TGF-beta, or any combination thereof.

10. The method of claim 1 wherein the migration channels of the microfluidic device have a width in the range of 2 to 50 μm, a height in the range of 1 to 20 μm, and a length in the range of 100 to 1000 μm.

11. The method of claim 1 wherein the microfluidic device is constructed of polydimethylsiloxane.

12. The method of claim 1 wherein the central chamber is filled with a gel to construct 3D culturing environment.

13. The method of claim 12 wherein the gel is either naturally isolated or synthesized and including: Matrigel™, Collagen, Gelatin, Hyaluronic Acid, Alginate, Polyethylene glycol (PEG), or any combination thereof.

14. The method of claim 1 wherein at least the base of the microfluidic device is coated with a biocompatible agent including: Collagen, Matrigel™, Poly-D-lysine, Poly-L-lysine, fibronectin, which facilitates cellular attachment to and migration on the device.

15. The method of claim 1 wherein the microglial cells in the central chamber are microglial cells activated by the chemoattractants or attractive cytokines and isolated from spontaneously activated microglia by culturing media in the annular chamber.

16. The method of claim 15 wherein the non-activated microglia and activated microglia are separated in a regulated manner based on microglia motility in response to the chemoattractants or attractive cytokines.
